(19) United States
(12) Patent Application Publication
(10) Pub. No.: US 2021/0196133 A1
(43) Pub. Date: Jul. 1, 2021

(54) SYSTEM FOR SUPPORTING A MOVEMENT EXERCISE OF A PERSON WITH AN OBJECT, METHOD AND COMPUTER PROGRAM PRODUCT

(71) Applicant: Fraunhofer-Geseilschaft zur Förderung der angewandten Forschung e.V, München (DE)

(72) Inventors: Thomas Von Der Grün, Kleinsendelbach (DE); Sylvie Couronne, Erlangen (DE)

(21) Appl. No.: 16/756,533

(22) PCT Filed: Oct. 26, 2018
(86) PCT No.: PCT/EP2018/079439

§ 371 Date: Apr. 16, 2020

(30) Foreign Application Priority Data
Oct. 27, 2017 (DE) 10 2017 125 189.8

(51) Int. Cl. A61B 5/0205; A61B 5/00; A61B 5/0531

(52) U.S. Cl. CPC A61B 5/02055; A61B 5/742; A61B 5/0531; A61B 2505/09; A61B 5/0004; A61B 5/681; A61B 5/0077; A61B 5/0075

(57) ABSTRACT

The present disclosure relates to a system for supporting a movement of a person with an object. The system comprises a detection device configured to detect an actual state of the object. The system also comprises a sensor arrangement, which is configured to record at least one physiological parameter of the person. The system also comprises a determination device configured to determine a target state of the object based on the detected physiological parameter. The system furthermore comprises a display device configured to display an indication when the actual state differs from the target state. The present disclosure further relates to a method and a computer program product.

### CROSS-REFERENCE TO RELATED APPLICATIONS

The present application is a national phase entry of PCT Application No. PCT/EP2018/079439, filed on 26 Oct. 2018, and entitled “System For Supporting A Movement Exercise Of A Person With An Object, Method, And Computer Program,” which claims the benefit of German Patent Application No. 10 2017 125 189.8, filed on 27 Oct. 2017, and entitled “System zur Unterstutzung einer Bewegungsübung einer Person mit einem Objekt, Verfahren un Computerprogrammprodukt.” These applications are incorporated herein by reference for all purposes.

### BACKGROUND

In sports, especially in competitive sports, the training of an athlete plays an increasingly important role. It is important to adapt the training to the athlete's actual performance. However, the athlete's actual performance can vary widely. The athlete's actual performance can depend on many different factors. In training, movement exercises are carried out to improve the condition, in particular endurance, strength, speed, mobility, but also to improve the technique of the athlete, in particular coordination and movement skills.

Objects can be used to improve the athlete's skills. For example, sport-specific objects are used, such as a soccer ball, a basketball or a volleyball for the sport of the same name or, for example, a boxing glove for boxing. Racquets can also be used in sports such as tennis, hockey or golf. Objects that are not sport-specific can also be used for training, such as a soccer ball for martial arts or a barbell for gymnastics.

With such objects, training exercises can be carried out in order to specifically train individual or multiple muscles or muscle groups. Other exercises with the objects serve to improve the technique of the athlete or other training goals. This is mainly about the optimization of training success through targeted additional workload and avoidance of underloading. Nevertheless, it can happen that the athlete is overwhelmed. Overworking the athlete can lead to an increased risk of injury.

The training of an athlete can be led by a trainer. However, it is often difficult for a trainer to correctly assess the athlete's actual performance and adapt the training accordingly.

### SUMMARY

It is therefore an object of the present disclosure to demonstrate an improved concept for supporting a person's movement exercise with an object.

According to a first aspect, the object is solved by a system for supporting a person's movement exercise with an object. The system comprises a detection device for detecting an actual state of the object. The system further comprises a sensor device for detecting at least one physiological parameter of the person. The system comprises a determination device for determining a target state of the object as a function of the detected parameter. The system also comprises a display device for displaying an indication of the target state of the object if the actual state differs from the target state.

The person can be an athlete, for example a competitive athlete who completes a training unit, in particular with individual training support. Alternatively, the person can be an animal to be trained. The object can be a training object, e.g. be a piece of sports equipment that the person uses. Alternatively, the object can be an animal with which the person trains a training exercise. For example, the object is a ball, for example a soccer ball, a basketball or a squash ball. The object can also be a racket, such as a hockey stick, tennis racket or golf club.

The detection device detects the actual state of the object. The current state of the object is the actual state of the object. The actual state can comprise a spatial and/or speed-dependent and/or acceleration-dependent component. The actual state can be detected relative to the person, to a system device and/or to a training room. The detection device can detect the actual state in a video-based and/or radio-based and/or optical manner. The detection device can comprise inertial sensors, such as acceleration sensors.

A sensor arrangement is provided in the system, which is set up to detect physiological parameters of the person. For this purpose sensors of the sensor arrangement and/or measuring modules are arranged distributed on the body of the person. They can be integrated in a textile, such as a training shirt, or worn in a belt or as a portable computer system, a so-called “wearable”, such as a fitness bracelet or a smart watch. The sensors can be connected via a communication module and thus send data and physiological parameters of the person. The sensor arrangement transmits the detected physiological parameters to the detection device. The sensors can also be connected by cable and thus transmit the physiological parameters of the person.

The detection device receives the physiological parameters of the person detected by the sensor arrangement and forwards them to the determination device.

The determination device can be part of the detection device. The determination device determines a target state of the object from the determined physiological parameters and predetermined determination rules and, if applicable, predetermined additional parameters. Additional parameters can be training parameters, in particular a type of training, training goals or target training intensity. They can comprise environmental parameters, in particular the time of day, temperature or size of a training area. They can also comprise individual static parameters of the person, for example as profile data such as gender, age, etc., but also a role in a team sport (e.g. goalkeeper or attacker or defender in football etc.).

The target state of the object is the state of the object that the object would have as the actual state if the movement were carried out correctly. This means that the target state is determined as the goal of the movement exercise and is dependent on a set training goal. For example, a target state is a hit target to train the person's accuracy or a frequency of the object within a certain range to train the person's endurance. Likewise, a deformation or an acceleration of the object can serve as the target state, for example to train a person's strength or his sprint speed.

The actual state of the object can also be used to determine the target state of the object. Likewise, further status data of further objects or further persons can be used, for example positions of teammates in a team sport. Likewise, the determination of the target state of the object can be based on a set of rules or guidelines, for example rules of the game or training rules, such as changes in pace, ball shooting techniques, stroke movement techniques, dribbling and others.

Algorithms can be used for this, which implement important elements of the set of rules or guidelines and analyze these elements to calculate the target state of the object. The elements are, for example, the definition of an “off” or a field size. In football, for example, a distinction is made between passes and long passes. These different training goals require different movements, which can be included as elements in the algorithms as parameters. For example, in addition to passes and long passes, a penalty situation when training shooting techniques is also differentiated. Each of these different game situations requires a different focus, such as shot power, shot accuracy or shot speed. In running exercises, a distinction can be made between sprinting, dribbling, dribbling in serpentine lines, etc. These training goals can be implemented as skill training with the ball and important parameters such as distance between foot and ball or running speed can be included in the algorithm as control elements. In tennis, a sequence of movements or another parameter of a backhand stroke, e.g. a length of the outward movement, when strength training with a barbell, can be used as the basis for a training rule.

The data can be processed by an analysis module, for example as part of the determination device or also another processor, in order to be output on the display device. The data can also be processed in the display device. The processing can comprise determining a difference between an actual status value and a target status value of the object. The difference can then be compared with a predetermined value, for example a threshold value. This threshold value corresponds to a measure of how much tolerance the difference between the actual state and the target state may have in order to count as a successfully achieved actual training target. This value can be set as an absolute number or as a percentage or depending on the physiological condition of the exerciser and can adapt accordingly. Any change in the threshold value can have an impact on the training goal, for example triggering the suggestion to take a break or changing the duration of the training session.

Results of the processing and further information can be displayed on the display device. The display device can be, for example, virtual reality glasses, or a monitor or a computer system such as a smart watch, a tablet computer or other mobile devices such as smartphone. In addition, vital parameters, that is, display parameters of the exerciser or display parameters determined by the trainer, can be output. Furthermore, a difference between the actual state value and the target state value of the object can be output. This can, for example, be performed by outputting a length difference if the actual state and the target state comprise a spatial component. Furthermore, this can be done as output of a force difference if the actual state and the target state comprise an acceleration component or a force component.

The display can be quantitative or qualitative. The display can include one or more indications if the difference between the actual state value and the target state value exceeds a predetermined threshold value. Here, a reference difference value between the actual state value and the target state value of the object can be read out, which is stored, for example, as a predetermined threshold value in a database. The display device can be integrated in the detection device or in one of the other devices. The display device can be wired or wirelessly connected to the remaining components of the system.

The display device can be connected to the system via a data network, for example via the internet. The system can have multiple display devices that display information in multiple different locations. The same content can be displayed here. However, different information can also be displayed for different people, such as the athlete and the trainer. For example, information is presented in full to a trainer and information is presented to the person, that is to say the exerciser, to a lesser extent. Likewise, the information that is presented to the trainer can be presented to the person in a clear and easy to understand manner. An avatar of the person can be shown as an animation figure on the display device in order to make the training instructions more understandable.

One advantage of such a system is that the person can be shown current data on their movement exercise in order to adapt and control their movement exercise. The information returned is based on objective measurement data that take into account the athlete's actual performance and environmental conditions. A training can thus be individually adjusted depending on the person's actual performance. The adjustment can be done automatically by the system, so that a trainer does not have to make any changes and adjustments manually during a training session. However, the system still allows manual intervention by the trainer or another person. This can be done using an input field in the display module. Here, the trainer can influence the training goals or the design of the training unit and, if necessary, instruct changes to the training process in the training process that are based on the objective information shown in the display module.

According to an advantageous example, the object is adapted to transmit a position signal, in particular a radio signal. The detection device is adapted to receive the position signal and to determine the actual state of the object on the basis of the received position signal.

The detection device can have a communication interface in order to detect the position signal. The communication interface can in particular be WLAN (for example WiFi), Zigbee, Bluetooth, UWB, UHF-RFID. Here, the position of the object, for example from a sensor in the object, can be transmitted to the detection device. Inertial sensors can be arranged in the object.

This enables wireless communication between the object and the detection device. This facilitates data transmission from the object to the detection device.

According to an advantageous example, the sensor arrangement is adapted to record at least one of the following physiological parameters: vital parameters, in particular heart rate, respiratory rate, oxygen concentration, blood sugar value, blood pressure, skin resistance, myoelectric activity, brain electrical activity, and/or biomechanical parameters, in particular a time parameter biokinematic parameter or a biodynamic parameter.

The sensors for this can include the following sensors: pulse sensor, pulse oximeter, pressure sensor, ECG, breathing air measuring device, piezoelectric sensors, strain gauges. The sensors can also be adapted to detect the following parameters: sweat gland activity, phasic and/or tonic changes, loss of minerals, calorie consumption, energy requirements, metabolic effectiveness, sugar or fat burning, cell oxygen absorption, muscle activity. Determining the target state of the object based on such values can increase the accuracy of the system, to specify a suitable movement exercise depending on the person's actual performance.

According to an advantageous example, the sensor arrangement is adapted to detect the physiological parameter of the person in a contactless or touch-based manner.

Touch-based detection can be done reliably and quickly. An advantage of contactless detection is that contactless detection can be more ergonomic.

According to a further advantageous example, the sensor arrangement is adapted to record an image of the person, in particular an infrared image, to detect at least one physiological parameter based on the image.

Image acquisition is a non-invasive acquisition method that can acquire physiological parameters without affecting the person. If it is, for example, an infrared camera, then infrared images can be recorded which, for example, record a body temperature.

According to an advantageous example, the sensor arrangement has a sensor for detecting the physiological parameter, the sensor being arranged in the object.

The sensor can be a sensor that can detect physiological parameters. For example, the sensor detects a mineral loss or an electrolyte content of a body fluid, for example via an analysis of sweat or also a temperature or oxygen saturation of the person. The sensor can be arranged in the object. The object can be a racket, such as a hockey stick, a tennis racket, a golf club, but also a ball that is held in one hand. Here, the object is adapted in such a way that, in order to record the physiological parameter, it brings the sensor into contact with or near the person in order to be able to record the physiological parameter.

An advantage of this configuration is that the ergonomics is increased further, since no additional sensor has to be attached directly to the body of the person, but rather the sensor can come into contact with the body and the person when they touch the object.

According to a further advantageous example, the sensor arrangement is adapted to transmit the detected physiological parameter to a determination device wirelessly, in particular by WLAN, Zigbee, Bluetooth, UWB, UHF-RFID, or by wire.

A wireless transmission has the advantage that the transmission can be carried out over a greater distance and very ergonomically and allows more freedom of movement. A wired transmission can have interference-free transmission, is easier to implement and cheaper.

According to an advantageous example, the determination device is adapted to assign the target state to the physiological parameter detected.

By assigning the target state to the recorded physiological parameter, a dynamic goal can be achieved in training. For example, the target state can be adjusted as the person's effort increases and the training duration increases and, if necessary, a training target to be achieved can be simplified.

According to an advantageous example, the determination device comprises a database in which different target states or differences between target states and actual states different are assigned to physiological parameters as predetermined threshold values.

The differences determine a threshold value that can be easily evaluated for an evaluation. Using a database makes using the system easier. Calling up data records from the database can take place quickly and thus accelerate the determination of the target state and/or the display on the display device.

The difference between the target state and the actual state can be performed during operation of the system by an analysis module which is integrated in the determination device or the display device or can also be arranged separately from these. On the basis of the threshold value determined and a comparison with threshold values or target states stored in the database, it can be decided which indications the display device should display.

According to an advantageous example, the determination device is adapted to determine the target state in a further dependence on a movement state of the object, in particular on an object speed or on an object acceleration.

This enables dynamic objectives to be set. For example, the target state of the object can take place depending on the speed. It is often important in sports to accelerate an object or bring it to a certain speed. This then influences the target state of the object, and depending on the adjusted target state, it can be shown that the acceleration or the speed was too slow or too high. A force, for example an impact force or a throwing force, can also be determined in this way.

According to an advantageous example, the determination device is adapted to additionally determine the target state as a function of at least one of the following parameters: age of the person, performance characteristics of the person, training intensity level, training duration, selection of targeted training units.

The performance of an athlete depends not only on his actual performance and on his actual physiological parameters, but also on other parameters relating to the person, such as the parameters mentioned above. It is also crucial which objectives and which requirements should be achieved through the training. Adaptation to such an objective, taking into account the parameters mentioned, can improve a movement exercise. Examples of a training intensity level is an intensity for one of the following training courses: performance training, warm-up training, rehabilitation training, physiotherapy training, endurance training.

According to an advantageous example, the determination device is adapted to determine the target state of the object as a further function of an actual state or target states of a further object.

Different objects can be important for a training, for example a golf ball and a hole for punching the golf ball. Another example is a soccer ball and a soccer goal or a soccer goal wall. Such constellations can be described for many sports. A system adapted in this way can, for example, improve target accuracy. Such a configuration can also be advantageous in team sports.

According to a further advantageous example, the determination device comprises a communication interface which is adapted to set up data transmission from the determination device to a computer system.

The computer system can comprise the detection device and/or the sensor device and/or the display device. The computer system can also be a server on which training data is stored. Data, such as display data or control data, can thus be passed on by the determination device via the communication interface. The communication interface includes, for example, a radio module for wireless data transmission or a data network connection for connecting to a data network, for example the internet.

According to a second aspect of the present disclosure, the object is solved by a method for supporting a person's movement exercise with an object. The method includes the following steps:

Detecting an actual state of the object by a detection device;

Detecting at least one physiological parameter of the person by a sensor arrangement;

Determining a target state of the object as a function of the detected physiological parameter by a determination device; and

Displaying an indication of the target state of the object on a display device if the actual state differs from the target state.

According to a third aspect, the object is solved by a computer program product according to claim 17.

### BRIEF DESCRIPTION OF THE DRAWINGS

The present disclosure is explained in more detail below on the basis of further examples and the figures. They show:

FIG. 1 is a schematic representation of a system according to an example of the present disclosure;

FIG. 2 shows a schematic representation of a system according to a further example of the present disclosure; and

FIG. 3 shows a schematic representation of a flow diagram for a method according to an example of the present disclosure.

### DETAILED DESCRIPTION

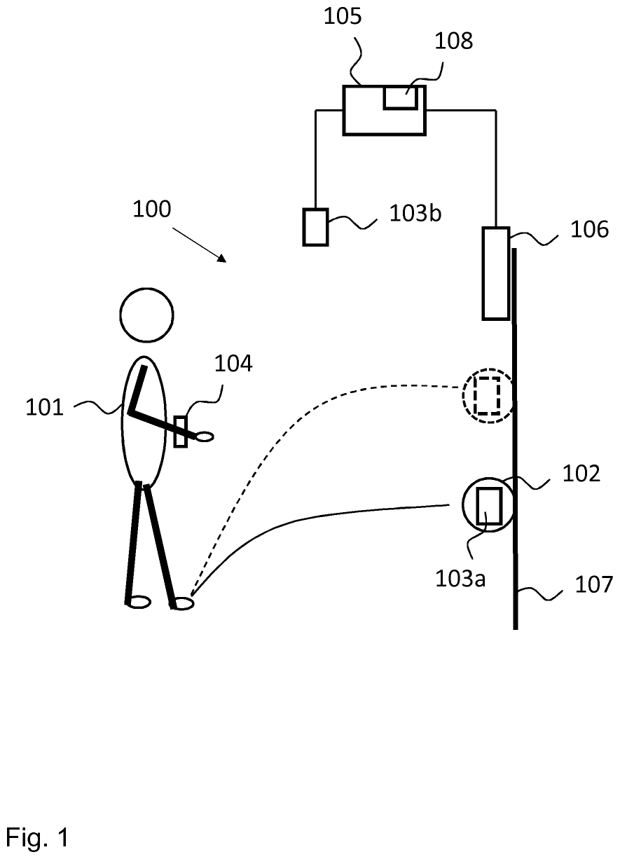

In FIG. 1 a person 101 is shown. The Person 101 trains a sport, here football. FIG. 1 shows a system 100 according to a first example. The system 100 comprises an object 102. In the exemplary example, the object 102 is a soccer ball. Furthermore, a goal wall 107 is shown against which the person 101 should shoot the object 102. For this purpose, the goal wall 107 has a target (not shown). In a further exemplary example, the object can be a boxing glove, which is used, for example, in boxing.

In alternative configurations, the person 101 does not train soccer, but a different sport. In this case, the object 102 is an object adapted to the other sport. In this alternative example, the goal wall 107 can be omitted or can be adapted accordingly to the sport.

The system 100 comprises a detection device 103. In the example shown, the detection device 103 is divided between two different sensors. First, a first sensor 103a is attached to the object 102.

The first sensor 103a is an inertial sensor, in particular an acceleration sensor. The detection device 103 also comprises a second sensor 103b. In the example shown, this is a camera. The second sensor 103b is adapted to optically detect the object 102. The first sensor 103a is adapted to detect the object 102 on the basis of acceleration values. An actual state of the object 102 can be determined from the two values of the different sensors 103a, 103b of the detection device 103. In the example shown, the first sensor 103a has a radio module in order to send the measured data to the detection device 103. The detection device 103 comprises a radio module which is adapted to receive radio data which is transmitted by the first sensor 103a. In a further exemplary example, a plurality or all of the sensors of the detection device 103 have a radio module in order to send the respectively recorded data to the detection device 103.

For this purpose, data from the first sensor 103a and data from the second sensor 103b are evaluated. The data from the acceleration sensor can supplement the image data from the camera. In a further exemplary example, the detection device 103 additionally or alternatively comprises a plurality of cameras and/or a plurality of acceleration sensors and/or other sensors, such as, for example, an optical system with lasers. The detection device 103 determines the actual state, i.e. the actual state of the object 102 in the training room in which the person 101 trains and the system 100 is arranged, for example the position of the object 102. In a further exemplary example, the object 102 comprises a position sensor and emits a position signal to determine the position of the object 102. The detection device 103 has a radio sensor that receives the position signal from the object 102 and uses this to determine the actual state.

The system 100 also has a sensor arrangement 104. In the example shown, the sensor arrangement 104 is arranged on the person 101. In the example shown, the sensor arrangement 104 is a fitness tracker which is worn on the wrist of the person 101. In addition, a belt can be used to which at least a part of the sensor arrangement 104 is fastened, for example a chest belt. In further refinements, the sensor arrangement 104 can comprise other and/or additional sensors, for example sensors woven into a textile for wearing as a T-shirt or also adhesive electrodes. The sensor arrangement 104 is adapted to detect at least one physiological parameter of the person 101. In the example shown in FIG. 1, the sensor arrangement 104 is adapted to detect the oxygen content of the blood, the temperature and the pulse of the person 101. In further configurations, further physiological parameters can be recorded. For example, vital parameters, in particular heart rate, respiratory rate, blood sugar value, blood pressure, skin conduction resistance, myoelectric activity, brain electrical activity, and/or biomechanical parameters, in particular a time parameter, a biokinematic parameter or a biodynamic parameter, are recorded for this purpose.

The system 100 also comprises a determination device 105. In the example shown, the determination device 105 is a computer system, in particular a microcontroller, which is arranged separately in the training room in which the person 101 trains. In further examples, the determination device 105 can be arranged in the detection device 103 or the sensor arrangement 104 or in a display device 106, or can also be set up as a virtual system on a server which is connected to the system 100 via a data network connection.

The determination device 105 is set up to receive the detected physiological parameters of the person 101 from the sensor arrangement 104. For this purpose, the sensor arrangement 104 can be connected to the determination device 105 in a wireless or wired manner. In the example shown, the sensor arrangement 104 is wirelessly connected to the determination device 105. A Bluetooth radio standard is used for this. In other examples, another wireless communication takes place between the sensor arrangement 104 and the determination device 105, for example using WLAN, Zigbee, UWB, UHF-RFID. In a further example, transmission takes place wirelessly and by cable, the physiological parameters recorded in each case being transmitted from different sensors to the person 101.

The determination device 105 is also connected to the detection device 103. Via the connection to the detection device 103, the determination device 105 can receive the detected actual state of the object 102 from the detection device 103. In the examples shown, the determination device 105 is connected wirelessly to the first sensor 103a and wired to the second sensor 103b. In a further example, the determination device 105 is not directly connected to the detection device 103. In this case, the actual state is transmitted by a further computer system or a separate microcontroller, for example a separate analysis module.

The determination device 105 determines a target state of the object 102. The target state of the object 102 can be a target of the object 102, such as the target on the goal wall 107. If the detection device 103 detects that the object 102 reaches the target state, i.e. here the target on the goal wall 107, then the actual state of the object 102 corresponds to the target state of the object 102.

If the person 101 shoots the football, the object 102, however not precisely enough, the object 102 hits the goal wall 107 at a different location than the target. The target state of object 102 is shown in dashed lines in FIG. 1. The actual state of the object 102 is shown in solid lines in FIG. 1. In FIG. 1, the actual state is different from the target state.

The actual state of the object 102 thus differs from the target state of the object 102. This is recognized by comparing the actual state of the object 102 with the target state of the object 102. For this purpose, the actual state of the object 102 and the target state of the object 102 are determined as a data comparison by an analysis module 108. The analysis module 108 is arranged in the determination device 105 and is implemented as software. In a further example, the analysis module 108 is arranged as hardware and/or in one of the other devices of the system 100 or else as a separate device.

The analysis module 108 is connected to a database via a data network connection. The analysis module 108 evaluates the data on the actual state acquired by the detection device 103, the physiological data of the person training and the target state of the object 102. In addition, the analysis module 108 has access to data for training purposes and training specifications from the database and can furthermore access predetermined stored target state values and use them to calculate a threshold value especially for the training person. In a further example, the analysis module has access to characteristic physiological values of the exercising person, for example in the resting state, in different effort levels, etc., or to typical physiological reference values of a sample person with a similar profile.

To determine the target state of the object 102, the determination device 105 evaluates the detected physiological parameters of the person 101. For this purpose, the physiological parameters of the person 101 are sent from the sensor arrangement 104 to the determination device 105. If the person 101 has a very high pulse and low oxygen saturation, the target value of the object 102 has a large tolerance. In a further example, an easily attainable target state is specified in addition or as an alternative to the tolerance. With such physiological parameters, the person 101 can only fire a targeted shot with difficulty. This is then recognized by the determination device 105 and the target state of the object 102 is determined accordingly, so that the target state of the object 102 can be achieved more easily by the person 101 by shooting the object 102. This means that if the physiological parameters indicate excessive physical exertion, an easily attainable target state value or a high threshold value is specified. Additionally or alternatively, an indication of a break can also be specified.

In a further example, the determining device 105 specifies a shot force or an acceleration of the object 102 as the target state of the object 102. Here, the acceleration data of the first sensor 103 of the sensor arrangement 103 can be evaluated.

If, on the other hand, the physiological parameters of the person 101 are high, that is to say the detected physiological parameters indicate a low effort, a target state that is difficult to achieve, or a low threshold value, is specified in order to keep the training target high. This specification can be made by the determination device 105 or by the analysis module 108. Alternatively, it can pass on an indication of a higher training target and/or longer training duration on the display device 106.

The system 100 also comprises the display device 106. The display device 106 is a flat screen monitor in the example shown. In a further example, it is a loudspeaker for outputting acoustic signals. Another option is to use vibration signals to specifically alert the trainee to a specific event, such as an inadequate movement or impending overexertion. The display device 106 is arranged separately in the example shown. In a further exemplary example, the display device 106 is integrated in the detection device 103. The display device 106 is adapted to display an indication of the target state of the object 102. In the example described for FIG. 1, the display device 106 shows whether the object 102 has hit the target in the goal wall 107 or not. Did the person 101 shoot the object 102 so well that the target was hit, i.e. that the target state of the object 102 corresponds to the actual state of the object 102, a positive result is displayed on the display device 106, for example a green color or a smiling smiley face or a success score.

However, if the target state of the object 102 does not match the actual state of the object 102, this is also displayed on the display device 106. For example, the display device 106 displays an arrow pointing in the direction in which the object 102 has to be shot the next time the person 101 tries. In another example, it can be displayed on the display device 106 that the object is to be struck firmer, or that a negative output takes place, such as a red color or a negative smiley face or a negative success score.

The system allows the person 101 to be trained to deal with the object 102 more precisely and to meet the target state of the object 102 better and more precisely over several training cycles. Furthermore, the system 100 can be used to ensure that the person 101 completes an endurance training session in which the person 101 is given training values via the display device 106 which result in that the person 101 plays the object 102 more frequently, faster and/or more vigorously. The information displayed on the display device 106 depends on the physiological parameters of the person 101 detected by the sensor arrangement 104. This allows the system 100 to adjust the target state of the object 101, depending on how busy, concentrated or fit the person 101 is.

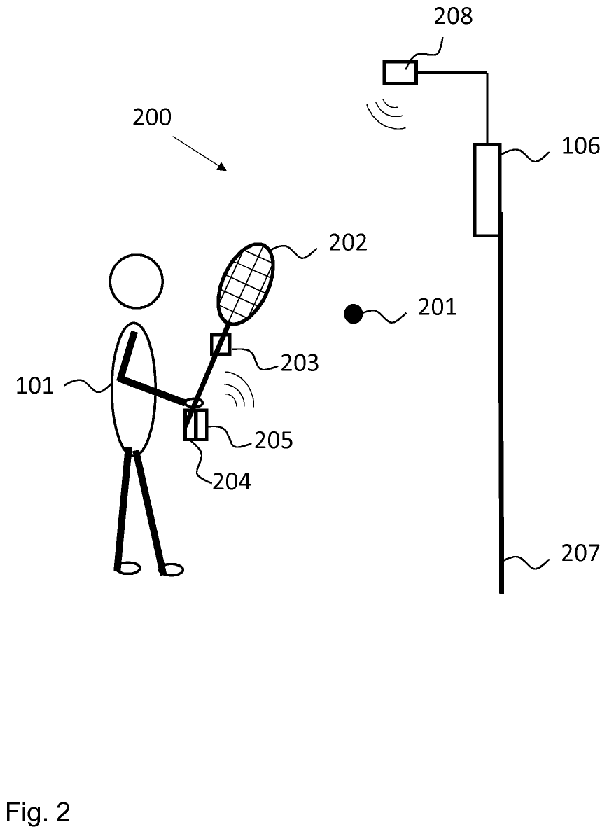

FIG. 2 shows a system 200 according to a further example. In the example shown in FIG. 2, the person 101 is a tennis player. The tennis player holds the object 102, here a tennis racket, in his hand. A tennis ball 201 is to be hit with the object 102. The tennis ball 201 is to be struck against a ball wall 207. The ball wall 207 can have markings that are intended to simulate a tennis net.

The system 200 according to FIG. 2 comprises an object 202, here a tennis racket and a detection device 203. The detection device 203 is attached to the object 202. In the example shown in FIG. 2, the detection device 203 is a motion sensor that can detect the speed and the acceleration of the object 202. Data can thus be collected about how fast and how dynamically the person 101 is swinging the object 202. The system 200 further comprises a sensor arrangement 204. The sensor arrangement 204 is arranged in or on the handle of the object 202. The sensor arrangement 204 is arranged such that the person 101 is in contact with the sensor device 204 when holding the object 202. The sensor arrangement 204 can thus read out data about the physiological parameters of the person 101 from the contact with the hand of the person 101. For this purpose, the sensor arrangement 204 comprises electrodes via which a skin conduction resistance, the temperature and the pulse of the person 101 can be detected.

The system 200 also has a determination device 205. In the example shown in FIG. 2, the determination device 205 is arranged in the object 202. This is, in particular, a microcontroller that takes over the determination of the target state of the object 202. In a further example, the determination device 205 can also be arranged separately, as described, for example, in relation to FIG. 1. The determination device 205 evaluates the physiological parameters of the person 101, which were detected by the sensor arrangement 204. From this and from stored data records, such as previous physiological parameters of the person 101 and reference values for predetermined movements of the object 202, the determination device 205 determines the target state of the object 202.

The system 200 furthermore comprises an analysis module 208, which is arranged separately and evaluates the detected actual state of the object 202 by the detection device 203 and the target state of the object 202 by the determination device 205. Depending on the physiological parameters and other boundary conditions, such as time of day, temperature of the environment or a training cycle, the evaluation can determine a difference between the values of the target state of object 202 and the values of the actual state of object 202.

For example, the actual state of the object 202 is characterized by a first parameter, here a first speed value. In this example, the target state of object 202 is characterized by a second parameter, here a second speed value. If the difference between the first parameter and the second parameter is less than a predetermined threshold value, the actual state of the object 202 and the target state of the object 202 are regarded as the same, and this is output on the display device 206. However, if the threshold is exceeded, i.e. if the actual state of the object 202 and the target state of the object 202 are not the same, or if the actual state value differs from the target state value by more than a predetermined tolerance, a message is output on the display device 106. For example, it can be output here that the racket, i.e. the object 202 has to be swung faster or more firmly.

In a further alternative or additional example, the sensor arrangement 204 comprises a separately arranged camera (not shown in FIG. 2) for taking an image of the person 101, in particular an infrared camera for taking an infrared image. In this case, the physiological parameters of person 101 are recorded in the image, so a temperature of person 101 can be determined.

In other configurations, other sports can be trained. For example, similar to the example of FIG. 2, the system 200 is set up in such a way that hockey can be trained with a hockey stick or golf with a golf club. Examples similar to the system 100 shown in FIG. 1 can be used for rugby, other ball games, throwing games or even skiing.

In a further example, in addition or as an alternative to the detection of the object 202 according to the example in FIG. 2, the tennis ball 201 is detected as object 202 or as a further object. Here, the actual state and the target state are evaluated in accordance with the evaluation as described for FIG. 1.

According to further refinements, the sensors and devices of the system 100 and the system 200 are combined and/or comprise further sensors for detecting the physiological parameters and/or the actual state of the object 102, 202. In particular the arrangement of the parts of the systems 100, 200 described in FIG. 1 and FIG. 2 are interchangeable or combinable. This also applies to the described communications between the described parts of the respective system 100, 200. Data can thus be transmitted from the sensor arrangement 204 to the determination device 205 in the system 200 in the manner described for FIG. 1. The same applies to the further transmissions of data and signals in the respective systems 100, 200.

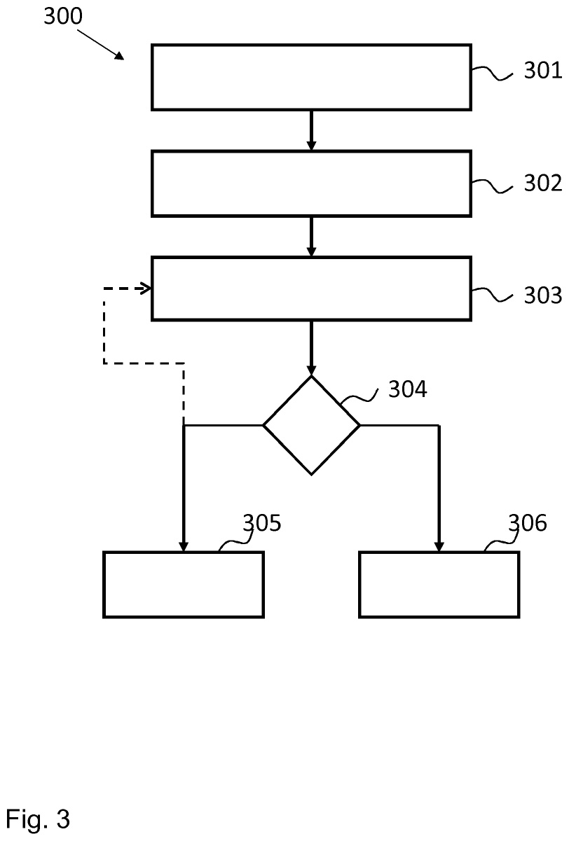

FIG. 3 shows a schematic illustration of a flow diagram 300 for a method according to an example of the present disclosure.

In a first step 301, the actual state of the object 102, 202 is determined. The detection of the actual state is carried out here by the detection device 103, 203, which determines the object 102, 202 using image data from a camera and/or sensor data from one or more sensors, such as acceleration sensors. Additionally or alternatively, the actual state can also be determined by a laser-based detection system.

In a further step 302, physiological parameters of the person 101 are determined. To determine the physiological parameters of person 101, data from sensor arrangement 104, 204 are used. Alternatively, step 302 can be carried out parallel to step 301 or step 301 before step 302.

In order to record the physiological parameters by the sensor arrangement 104, 204, measured values are recorded by one or different sensors. Thus, the determination device 105, 205 can pass on various physiological parameters, such as vital parameters, in particular heart rate, respiratory rate, oxygen concentration, blood sugar value, blood pressure, skin resistance, myoelectric activity, brain electrical activity, and/or biomechanical parameters, in particular a time parameter, a biokinematic parameter or a biodynamic parameter.

In step 303, a target state of the object 102, 202 is determined. The target state of the object 102, 202 is determined as a function of the physiological parameters recorded in step 302. For this purpose, the sensor arrangement 104, 204 sends the detected physiological parameters of the person 101 to the determination device 105, 205. The transmission can take place wirelessly or by cable. In the determination device 105, 205, the recorded physiological values of the person 101 can include predetermined information in order to be able to correctly interpret the physiological measured values in the analysis module 108, 208. The predetermined information is, for example, the age of the person and/or the gender and/or a disability and/or their own physiological reference values.

The determination device 105, 205 has access to a database in which reference values for target states and rules for determining the target state are stored. For example, such a rule includes a game rule of a sport to be trained.

The target state of the object 102, 202 comprises a form of movement and/or a speed and/or an acceleration of the object 102, 202 and/or a spatial position in the training room.

The target state can be determined via a data analysis platform. For this purpose, the system 100, 200 has corresponding communication means in order to exchange data with the data analysis platform.

In step 304, the recorded physiological values of the person 101, 201 are interpreted on the basis of predetermined information by the analysis module 108, 208, for example no noticeable effort is detected or that too many minerals have been lost, for example by sweating.

In step 304, the analysis module 108, 208 calculates a difference value between the actual state of the object 102, 202 and the target state of the object 102, 202. For this purpose, for example, two acceleration values are subtracted from one another or two spatial coordinates of the object 102, 202 are set in relation to one another. The result of the comparison is checked against a reference value. This reference value can be a threshold value which can be exceeded or fallen below. The threshold value can be absolute, e.g. be defined as a numerical value, but also relatively as a percentage. The threshold value can also be set dynamically and depend on other factors. This means that the threshold value can be varied depending on the physiological parameters, for example to make training easier when the person is under a lot of effort or when he is not concentrating, or vice versa.

If the threshold is exceeded, i.e. if the difference between the actual state of the object 102, 202 is greater than a predetermined difference value from the target state of the object 102, 202, the method is continued in step 305.

If the training flow changes (e.g. becomes more intensive), these measures can flow back into the determination device 105, 205 in order to update the rule for determining the next target state. This creates an automatic control loop (represented by the dashed arrow in FIG. 3). Additionally or alternatively, this regulation can run via the display device 106 in order to be confirmed or rejected by the exercising person and/or by the trainer (not shown in FIG. 3 for reasons of clarity).

In step 305, an indication is output on the display device 106, which indicates a desired target state of the object 102, 202. The indication is generated by the analysis module 108, 208. For example, the display device 106 indicates that the object 102, 202 is to be struck faster, further or more firmly, or it can also be indicated that the object 102, 202 should be played more precisely. Training measures can also be specified, such as a relaxation break, a drinking break or sharper or more intensive training.

If the threshold value is not exceeded, the method is continued in step 306. In step 306 there can be a positive output, for example a praise that the training goal has been reached. In a further example, no display is output in step 306.

The display device 106 can use an indication catalog for outputting the indication, in which predetermined indications are stored. The display device 106 can be used to input predetermined information as well as to confirm or reject suggested indications and to manually control the training process.

### LIST OF REFERENCE NUMBERS

100, 200 system

101 person

102, 202 object

103, 203 detection device

103a first sensor

103b second sensor

104, 204 sensor arrangement

105, 205 determination device

106 display

107 goal wall

207 ball wall

108, 208 analysis module

300 flowchart

301-306 method step

What is claimed is:

1. A system to support a movement exercise of a person with an object, comprising:
a detection device configured to detect an actual state of the object;
a sensor arrangement configured to detect at least one physiological parameter of the person;
a determination device configured to determine a target state of the object based on the detected physiological parameter; and
a display device configured to display an indication if the actual state differs from the target state.

2. The system according to claim 1, wherein the indication that is displayed by the display device indicates the target state of the object.

3. The system according to claim 1, wherein the object is configured to transmit a position signal, and wherein the detection device is adapted to receive the position signal and determine the actual state of the object based on the received position signal.

4. The system according to claim 1, wherein the sensor arrangement is configured to detect at least one of the following physiological parameters: heart rate, respiratory rate, oxygen concentration, blood sugar value, blood pressure, skin conduction resistance, myoelectric activity, electrical brain activity, a time parameter, a biokinematic parameter, or a biodynamic parameter.

5. The system according to claim 1, wherein the sensor arrangement is configured to detect the physiological parameter without contact or contact-based.

6. The system according to claim 1, wherein the sensor arrangement is configured to record an infrared image of the person and to detect the at least one physiological parameter based on the recorded image.

7. The system according to claim 1, wherein the sensor arrangement has a sensor configured to detect the physiological parameter, and wherein the sensor is arranged in the object.

8. The system according to claim 1, wherein the sensor arrangement is configured to transmit the detected physiological parameter to the determination device wirelessly or by wire.

9. The system according to claim 1, wherein the determining device is configured to assign the target state to the detected physiological parameter.

10. The system according to claim 1, wherein the determination device has a database in which different target states or differences between target states and actual states are assigned to different physiological parameters.

11. The system according to claim 1, wherein the determining device is configured to determine the target state further based on a movement state of the object.

12. The system according to claim 1, wherein the determining device is configured to determine the target state further based on at least one of the following parameters: an age of the person, a performance characteristic of the person, a training intensity level, a training duration, or a selection of targeted training units.

13. The system according to claim 1, wherein the determining device is configured to determine the target state of the object further based on an actual state or target state of another object.

14. The system according to claim 1, wherein the determination device comprises a communication interface configured to set up a data transmission from the determination device to a computer system.

15. The system according to claim 1, further comprising an analysis module configured to perform a comparison based on one or more of: the detected actual state, the at least one detected physiological parameter, or the specific target state, and provide the indication to the display device based on the comparison.

16. A method for supporting a movement exercise of a person with an object, comprising:
detecting an actual state of the object by a detection device;
detecting at least one physiological parameter of the person by a sensor arrangement;
determining a target state of the object based on the detected physiological parameter by a determination device; and
displaying an indication on a display device if the actual state differs from the target state.

17. A computer program product comprising a non-transitory computer readable medium storing program code for supporting a movement exercise of a person with an object, the program code comprising instructions configured to cause a computer system to:
detect an actual state of the object at a detection device;
detect at least one physiological parameter of the sensor at a sensor arrangement;
determine a target state of the object based on the detected physiological parameter at a determination device; and
display an indication on a display device if the actual state differs from the target state.
